(19) United States
(12) Patent Application Publication
(10) Pub. No.: US 2019/0105503 A1
(43) Pub. Date: Apr. 11, 2019

(54) LEAD ANCHORS FOR ELECTRICAL STIMULATION LEADS AND SYSTEMS AND METHODS OF MAKING AND USING

(71) Applicant: Boston Scientific Neuromodulation Corporation, Valencia, CA (US)

(72) Inventor: Jacob B. Leven, Huntington Beach, CA (US)

(21) Appl. No.: 16/149,930

(22) Filed: Oct. 2, 2018

Related U.S. Application Data
(60) Provisional application No. 62/569,469, filed on Oct. 6, 2017.

(51) Int. Cl. A61N 1/375; A61B 17/34

(52) U.S. Cl. CPC A61N 1/37518; A61N 1/05; A61B 17/3468

(57) ABSTRACT

A lead anchor may include a deformable body defining a lead passageway for receiving a lead. A lead retainer may be formed from a rigid material that is disposed in the body and that includes first and second retaining elements disposed around a portion of the lead passageway. First and second tool lumens may extend between an outer surface of the body and the first and second retaining elements, respectively. The retaining elements may be biased to exert a compressive force against a portion of a lead disposed in the lead passageway to resist axial movement of the lead relative to the lead anchor. The lead anchor may be configured so that a tool inserted into the first and second tool lumens can separate the first retaining element from the second retaining element to facilitate insertion or removal of the portion of the lead from the lead passageway.

### CROSS-REFERENCE TO RELATED APPLICATIONS

This application claims the benefit under 35 U.S.C. § 119(e) of U.S. Provisional Patent Application Ser. No. 62/569,469, filed Oct. 6, 2017, which is incorporated herein by reference.

### FIELD

The present invention is directed to the area of implantable electrical stimulation systems and methods of making and using the systems and, in particular, to implantable electrical stimulation systems that include lead anchors for anchoring leads to patient tissue, as well as methods of making and using the leads, lead anchors, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to central body tissue.

### BRIEF SUMMARY

In some aspects, an apparatus may be a lead anchor having an elongated body formed from a deformable material. The body may have an outer surface and a longitudinal length with a first end and an opposing second end. The body may define a lead passageway configured to receive a lead. The lead passageway may extend along the entire longitudinal length of the body from the first end to the second end. A lead retainer may be formed from a rigid material and is disposed in the body around a portion of the lead passageway. The lead retainer may include first and second retaining elements. The portion of the lead passageway may be between the first and second retaining elements. A first tool lumen may extend along the longitudinal length of the body between the outer surface of the body and the first retaining element. A second tool lumen may extend along the longitudinal length of the body between the outer surface of the body and the second retaining element. The first and second retaining elements may be biased to exert a compressive force against a portion of a lead disposed in the lead passageway to resist axial movement of the lead relative to the lead anchor. The lead anchor may be configured so that a tool inserted into the first and second tool lumens can separate the first retaining element from the second retaining element to facilitate insertion or removal of the portion of the lead from the lead passageway.

In at least some embodiments, the first and second retaining elements are each formed as single-piece structures.

In at least some embodiments, the first retaining element defines a first tool engagement aperture in communication with the first tool lumen, and the second retaining element defines a second tool engagement aperture in communication with the second tool lumen. In at least some embodiments, the first tool engagement aperture is configured to receive a first probe of a tool extended along the first tool lumen, and the second tool engagement aperture is configured to receive a second probe of the tool extended along the second tool lumen. In at least some embodiments, the first retaining element includes a first lead engagement portion and the second retaining element includes a second lead engagement portion. The first and second lead engagement portions are disposed along the lead passageway.

In at least some embodiments, the first retaining element is formed as a first multi-piece structure and the second retaining element is formed as a second multi-piece structure. In at least some embodiments, the first multi-piece structure includes a first tool engagement piece and a first lead engagement piece with the first tool engagement aperture defined in the first tool engagement piece and the first lead engagement portion disposed along the first lead engagement piece; and the second multi-piece structure includes a second tool engagement piece and a second lead engagement piece with the second tool engagement aperture defined in the second tool engagement piece and the second lead engagement portion disposed along the second lead engagement piece.

In at least some embodiments, the first lead engagement portion includes a first concave cutout configured to extend around a portion of the circumference wall; and the second lead engagement portion includes a second concave cutout configured to extend around a portion of the circumferential wall circumferentially opposite to the portion of the circumferential wall around which the first concave cutout extends. In at least some embodiments, the first lead engagement portion includes first guide features disposed on opposing sides of the first concave cutout; and the second lead engagement portion includes second guide features disposed on opposing sides of the second concave cutout. The first and second guide features are collectively configured to prevent portions of the body from being pulled inwardly between the first and second retaining element when the first retaining element is separated from the second retaining element.

In at least some embodiments, the first and second tool lumens open to the outer surface of the body along the first end. In at least some embodiments, at least one of the first tool lumen or the second tool lumen extend along the entire longitudinal length of the body and are open to the outer surface of the body along both the first and second ends. In at least some embodiments, the first tool lumen terminates at, or in proximity to, the first tool engagement aperture of the lead retainer. In at least some embodiments, the second tool lumen terminates at, or in proximity to, the second tool engagement aperture of the lead retainer.

In some aspects, an anchoring arrangement may include any of the lead anchors described above and a tool. The tool may include a first probe configured to extend through the first tool lumen of the lead anchor body and the first tool engagement aperture of the lead retainer and a second probe configured to extend through the second tool lumen of the lead anchor body and the second tool engagement aperture of the lead retainer. In at least some embodiments, the anchoring arrangement further includes an electrical stimulation lead. The electrical stimulation lead includes electrodes. The lead anchor is configured to receive a portion of the electrical stimulation lead and removably retain the received portion of the electrical stimulation lead.

In other aspects, an implantable stimulation arrangement may include any of the lead anchors described above and an electrical stimulation lead. The electrical stimulation lead may include electrodes. The lead anchor may be configured to receive a portion of the electrical stimulation lead and removably retain the received portion of the electrical stimulation lead.

In still other aspects, an implantable stimulation system may include any of the lead anchors described above; an electrical stimulation lead that may include electrodes and that may be coupleable to the lead anchor; and a control module that may be coupleable to the electrical stimulation lead.

In some aspects, a method of anchoring a lead may include providing the lead anchor described above. An electrical stimulation lead may be advanced to a target stimulation location within a patient. A force may be applied to separate the first and second retaining elements of the lead retainer of the lead anchor. The applied force may counteract an opposing compressive force exerted by the lead retainer. The electrical stimulation lead may be inserted into the lead passageway and advanced relative to the lead anchor while applying the force to separate the first and second retaining elements. The applied force may be released to hold the electrical stimulation lead in position relative to the lead anchor by the compressive force exerted by the body of the lead anchor.

In at least some embodiments, applying a force to separate the first and second retaining elements includes: inserting a first probe of a tool through a first tool engagement aperture of the first retaining element of the lead retainer; inserting a second probe of the tool through a second tool engagement aperture of the second retaining element of the lead retainer; and using the probes to physically separate the first and second tool engagement apertures from one another. In at least some embodiments, applying a force to separate the first and second retaining elements includes using a ratcheting mechanism to facilitate maintaining the applied force to counteract the compressive force of the lead retainer for an extended period of time.

In at least some embodiments, inserting a first probe of a tool through the first tool engagement aperture includes advancing the first probe along the longitudinal length of the lead anchor within the first tool lumen; and inserting a second probe of a tool through the second tool engagement aperture includes advancing the second probe along the longitudinal length of the lead anchor within the second tool lumen.

In at least some embodiments, the method of anchoring a lead further includes anchoring the lead anchor to patient tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic perspective view of one embodiment of a lead anchor, according to the invention;

FIG. 2B is a schematic longitudinal, cross-sectional view of one embodiment of the lead anchor of FIG. 2A, according to the invention;

FIG. 3A is a schematic perspective view of another embodiment of a lead anchor, according to the invention;

FIG. 3B is a schematic end view of one embodiment of the lead anchor of FIG. 3A, according to the invention;

FIG. 4 is a schematic end view of one embodiment of a lead retainer suitable for placement within either the lead anchor of FIGS. 2A-2B or the lead anchor of FIGS. 3A-3B, according to the invention;

FIG. 5A is a schematic, transverse cross-sectional view of one embodiment of the lead retainer of FIG. 4 disposed in a lead anchor, the lead retainer disposed in a biased position suitable to retain a lead when the lead is inserted into a lead passageway defined in the lead anchor, according to the invention;

FIG. 5B is a schematic, transverse cross-sectional view of one embodiment of the lead retainer of FIG. 4 disposed in the lead anchor of FIG. 5A, the lead retainer disposed in an open position suitable to enable a lead to move axially along a lead passageway defined in the lead anchor, according to the invention;

FIG. 6 is a schematic end view of another embodiment of a lead retainer suitable for placement within the lead anchor of either FIG. 2A or FIG. 3A, according to the invention;

FIG. 7A is a schematic perspective view of one embodiment of a tool suitable for facilitating transition of the lead retainer of either FIG. 4 or FIG. 6 from the biased position of FIG. 5A to the open position of FIG. 5B, according to the invention;

FIG. 7B is a schematic, close-up perspective view of one embodiment of probes extending from one end of the tool of FIG. 7A, the probes suitable for insertion into tool lumens defined in the lead anchor of either FIG. 2A or FIG. 3A to access the lead retainer of either FIG. 4 or FIG. 6 to transition the lead retainer from a biased position to an open position, according to the invention;

FIG. 8A is a schematic perspective view of one embodiment of the lead anchor of FIG. 2A and a portion of the tool of FIG. 7A, the tool including probes suitable for inserting into tool lumens of the lead anchor and using to transition the lead retainer from the biased position of FIG. 5A to the open position of FIG. 5B, according to the invention;

FIG. 8B is a schematic longitudinal, cross-sectional view of one embodiment of the lead anchor and tool of FIG. 8A, according to the invention;

FIG. 9A is a schematic perspective view of one embodiment of probes of the tool of FIG. 8A inserted into tool lumens of the lead anchor of FIG. 8A, according to the invention;

FIG. 9B is a schematic longitudinal, cross-sectional view of one embodiment of probes of the tool of FIG. 9A inserted into tool lumens of the lead anchor of FIG. 9A, according to the invention;

FIG. 10 is a schematic perspective view of one embodiment of a portion of the tool of FIG. 7A that includes an optional ratcheting mechanism suitable for enabling the tool to be held in a position suitable for keeping the lead retainer in an open position until manually disengaged, according to the invention; and

FIG. 11 is a schematic overview of one embodiment of components of an electrical stimulation system, according to the invention.

### DETAILED DESCRIPTION

The present invention is directed to the area of implantable electrical stimulation systems and methods of making and using the systems and, in particular, to implantable electrical stimulation systems that include lead anchors for anchoring leads to patient tissue, as well as methods of making and using the leads, lead anchors, and electrical stimulation systems.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, peripheral nerve, or cardiac-tissue stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

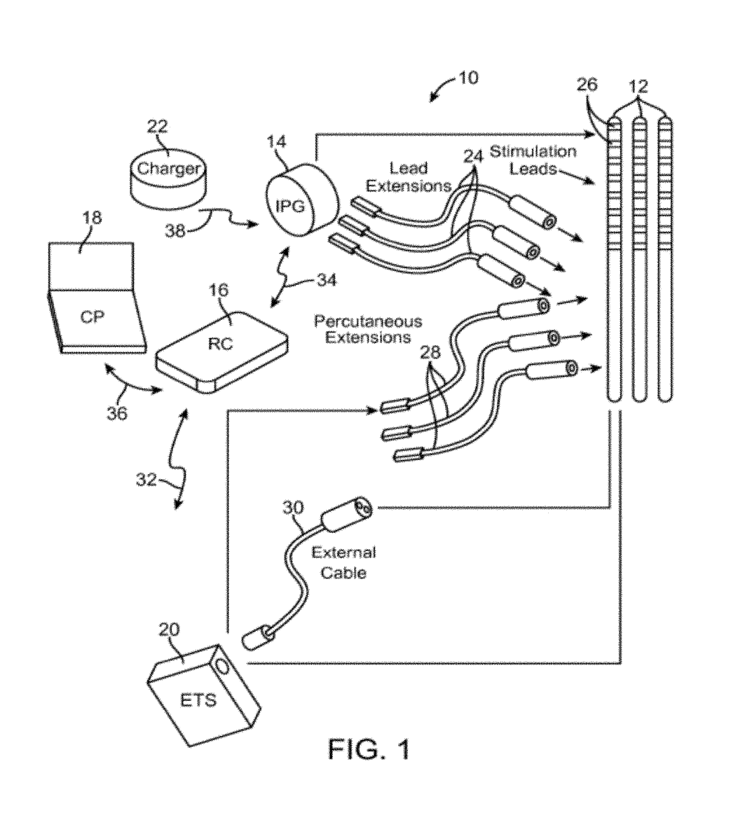

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's central body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

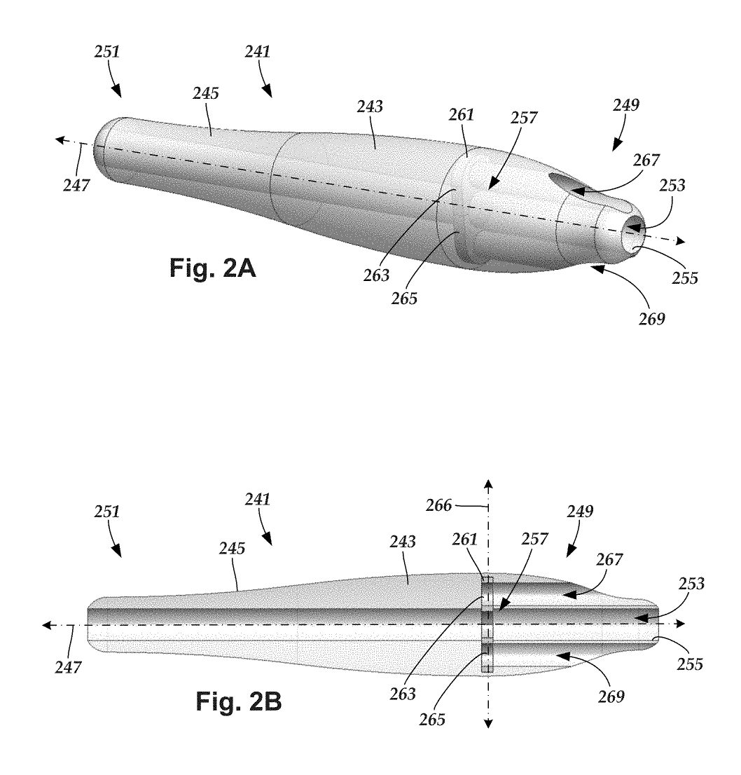

Turning to FIGS. 2A-2B, a lead anchor is described below. The lead anchor can be used in an implantable device, such as an implantable spinal cord stimulator or any other stimulator system, to anchor a lead or lead extension to patient tissue. The lead anchor will be described herein as anchoring a lead, but it will be understood that the lead anchor can also anchor a lead extension within the patient's body.

The below-described lead anchor uses a retaining element that is disposed in the lead anchor about a lead passageway and that is biased to exert a compressive force against a lead inserted into the lead passageway sufficient to cause the received lead to resist axial movement relative to the lead anchor until a counteracting force is applied to overcome the compressive force exerted by the lead retainer. This new design may reduce a profile of the lead anchor compared to conventional lead anchors along at least one lateral dimension. This new design may also reduce manufacturing costs, as compared to conventional lead anchors.

FIG. 2A illustrates, in schematic perspective view, one embodiment of a lead anchor 241. FIG. 2B shows the lead anchor 241 in longitudinal, cross-sectional view. The lead anchor 241 includes a deformable (e.g., elastic), elongated body 243 with an outer surface 245 and a longitudinal length 247 with a first end portion 249 and an opposing second end portion 251. The body 243 can be formed from any biocompatible, deformable material suitable for implantation (e.g., silicone, or other elastomer).

The body includes a lead passageway 253 that is defined by a circumferential wall 255 and that extends along the longitudinal length of the body. The circumferential wall, optionally, includes a compressible portion 257. In some embodiments, the compressible portion 257 is confined to one or more discrete regions within the body 243. In other embodiments, the circumferential wall is compressible along the entire longitudinal length of the body 243.

A lead retainer 261 is disposed in the body and positioned around the compressible portion of the lead passageway 253. Alternately, the lead retainer 261 can be disposed between two or more sections of the lead passageway 253. In which case, the lead retainer forms a portion of the lead passageway. The lead anchor will be described herein as including a single lead retainer, but it will be understood that the lead anchor can include multiple lead retainers. The lead retainer 261 is formed from a rigid material and is more rigid than the body. The lead retainer 261 is formed from a material that has a higher durometer than the body. In at least some embodiments, the lead retainer 261 is flat, or substantially flat.

The lead retainer 261 includes a first retaining element 263 and a second retaining element 265 disposed along opposing circumferential sides of the lead passageway 253. In at least some embodiments, the first retaining element 263 and the second retaining element are coplanar. In at least some embodiments, the first retaining element 263 and the second retaining element are coplanar along a plane 266 that is transverse to the longitudinal length of the body. In at least some embodiments, the first retaining element 263 and the second retaining element are coplanar along a plane 266 that is transverse to the longitudinal length of the lead passageway.

As will be described in more detail below, the lead retainer 261 is configured for transitioning from a biased position to an open position. In the biased position, the first and second retaining elements 263, 265 exert compressive forces into the lead passageway (or against the circumferential wall that defines the lead passageway) from opposing sides, thereby causing the lead retainer to impinge upon the lead passageway by an amount sufficient to cause a portion of a received lead to resist axial movement relative to the lead anchor.

The lead retainer is biased to the biased position. Application of a force in opposing directions is used to counteract the bias and transition the lead retainer to the open position. Application of a counteracting force, such as manual separation of the first retaining element 263 from the second retaining element 265, releases the compressive forces applied by the lead retainer and physically removes the impingement, thereby transitioning the lead retainer to an open position, where the lead can be moved axially along the lead passageway relative to the lead anchor. In at least some embodiments, the bias is from the deformable (e.g., elastic) body of the lead anchor. Accordingly, transition of the lead retainer to the open position (i.e., separating the first and second retaining elements) may involve stretching the deformable body along opposing directions transverse to the longitudinal length of the body, or the lead passageway, or both.

In some instances, it may be desirable to anchor the lead anchor 241 to patient tissue. Accordingly, the body 243 can, optionally, include one or more protruding features with eyelets (not shown) for receiving a suture, a staple, or the like, for securing the lead anchor to patient tissue. The protruding features may be circumferentially and axially disposed at any suitable location around the body 243. In at least some embodiments, the protruding features with eyelets are circumferentially offset by 180° from one another (or disposed on opposite sides of the body 243). In at least some embodiments, the protruding features are also axially, or longitudinally, offset from each other. The lead anchor can include any suitable number of protruding features including, for example, one, two, three, four, five, six, seven, eight, or more protruding features. The protruding features may be made from either the same material or different material from the body 243.

In at least some embodiments, the body 243 includes one or more optional suture channels (not shown) that are disposed at least partially around a circumference of the body 243. In at least some embodiments, a suture channel is also axially-aligned with one or more of the protruding features and eyelets. The suture channels facilitate suturing of the lead anchor 241 to patient tissue by enabling sutures to be disposed around the body 243 and passed through one or more of the eyelets without increasing the diameter of the lead anchor 241, while also preventing (or reducing the likelihood of) the sutures from slipping off an end of the body 243.

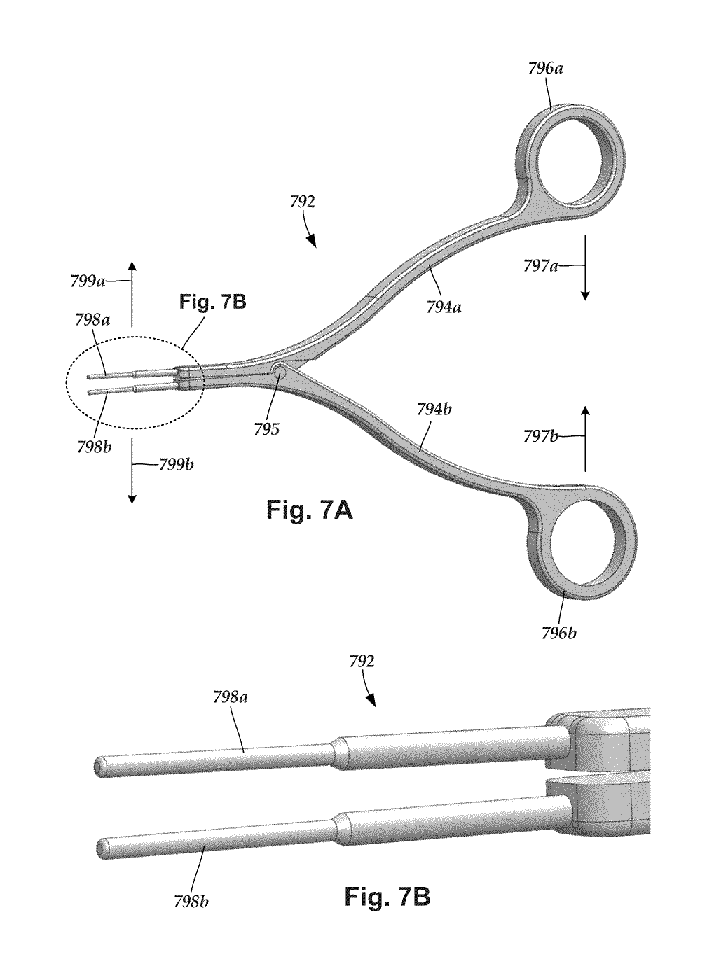

The body 243 defines a first tool lumen 267 extending between the outer surface 245 of the body 243 and the first retaining element 263 of the lead retainer 261. In at least some embodiments, the first tool lumen 267 extends along the longitudinal length of the body between the outer surface 245 of the body 243 and the first retaining element 263. Similarly, the body 243 defines a second tool lumen 269 extending between the outer surface 245 of the body 243 and the second retaining element 265 of the lead retainer 261. In at least some embodiments, the second tool lumen 269 extends along the longitudinal length of the body between the outer surface 245 and the second retaining element 265. The tool lumens 267, 269 provide access for a tool (e.g., 792 in FIG. 7A) to facilitate separation of the retaining elements 263, 265 to counteract the lead-retention bias of the lead retainer.

In at least some embodiments, the tool lumens 267, 269 extend parallel to one another along the body 243. In at least some embodiments, at least one of the tool lumens 267, 269 extends parallel to the lead passageway 253. In FIGS. 2A-2B the tool lumens 267, 269 extend from the first end portion 249 of the body and terminate at the lead retainer. In other embodiments, at least one of the tool lumens 267, 269 extends from the second end portion 251 of the body and terminates at the lead retainer.

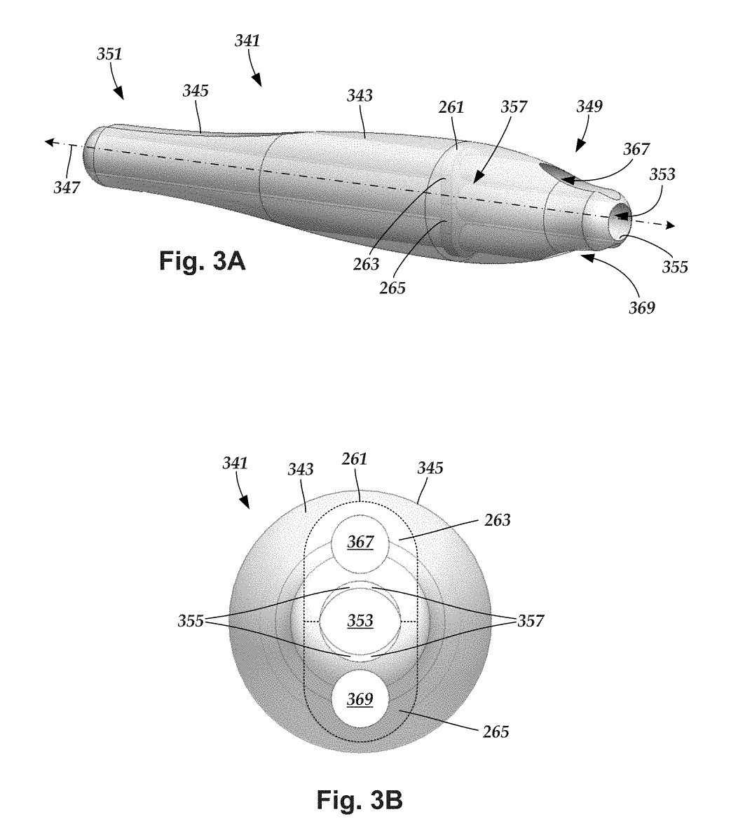

In at least some embodiments, at least one of the tool lumens extends along the entire longitudinal length of the body from the first end portion of the body to the second end portion of the body. FIG. 3A shows, in schematic perspective view, another embodiment of a lead anchor 341. FIG. 3B shows the lead anchor 341 in end view. The lead anchor 341 is similar to the lead anchor 241, described above. The lead anchor 341, however, differs from the lead anchor 241 in that each of the tool lumens of the lead anchor 341 extend along the entire longitudinal length of the body.

The lead anchor 341 includes a deformable (e.g., elastic), elongated body 343 with an outer surface 345 and a longitudinal length 347 with a first end portion 349 and an opposing second end portion 351. The body includes a lead passageway 353 that is defined by a circumferential wall 355 and that extends along the longitudinal length of the body. The circumferential wall includes a compressible portion 357.

The lead retainer 261 is disposed in the body and positioned around at least a portion of the compressible portion of the lead passageway 353. Alternately, the lead retainer 261 can be disposed between two or more sections of the lead passageway 253. In which case, the lead retainer forms a portion of the lead passageway. FIG. 3B shows the impingement of the circumferential wall 355 along the compressible portion 357 into the lead passageway 353 when the lead retainer is in the biased position and when there is not a lead inserted into the lead passageway. Alternately, FIG. 3B can be thought of as showing impingement of the lead retainer into the lead passageway 353 when the lead retainer is in the biased position and when there is not a lead inserted into the lead passageway.

The body 343 defines a first tool lumen 367 extending along the longitudinal length of the body from the outer surface 345 of the body 343 along the first end portion 349 to the outer surface 345 of the body along the second end portion 351 and extending through the first retaining element 263 of the lead retainer 361. Similarly, the body 343 defines a second tool lumen 369 extending along the longitudinal length of the body from the outer surface 345 of the body 343 along the first end portion 349 to the outer surface 345 of the body along the second end portion 351 and extending through the second retaining element 265 of the lead retainer 361.

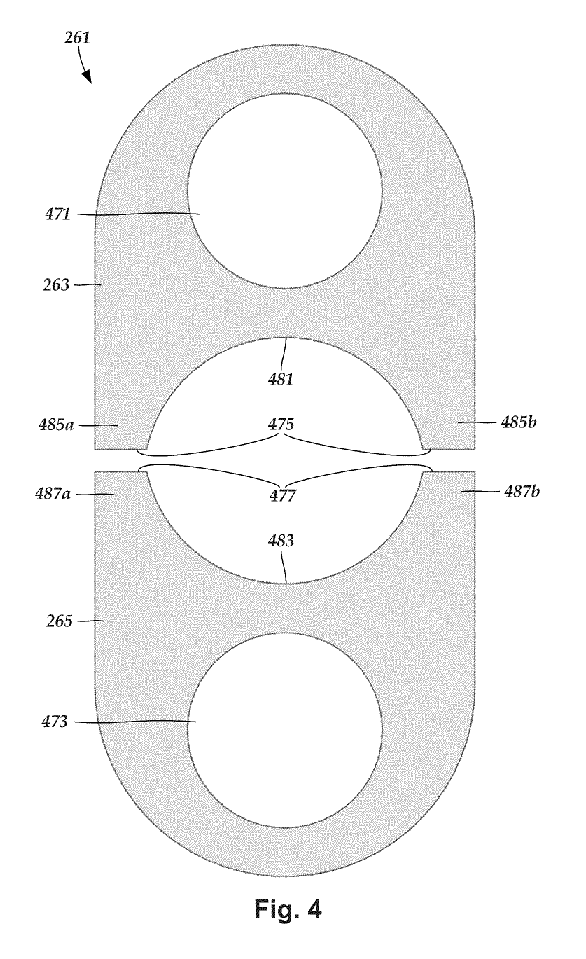

FIG. 4 shows, in schematic end view, one embodiment of the lead retainer 261. The lead retainer 261 is suitable for placement within either the lead anchor 241 or the lead anchor 341. As described above, the lead retainer 261 includes the first retaining element 263 and the second retaining element 265. In at least some embodiments, the retaining elements 263, 265 are flat, or substantially flat.

The first retaining element 263 defines a first tool engagement aperture 471 and the second retaining element 265 defines a second tool engagement aperture 473. The tool engagement apertures 471, 473 are configured to receive one or more tools suitable for separating the retaining elements 263, 265 from one another by an amount sufficient to enable a lead to be inserted or removed from the lead anchor, or enable a received lead to move axially along the lead passageway relative to the lead anchor. The tool engagement apertures 471, 473 are accessible to the tool via the tool lumens (e.g., 267, 269, respectively, of FIG. 2A-2B; or 367, 369, respectively, of FIG. 3A-3B), which extend between the tool engagement apertures and the outer surface of the body of the lead anchor.

In at least some embodiments, the retaining elements 263, 265 include lead engagement portions 475, 477, respectively, that are configured for positioning within the body of the lead anchor around opposing sides of the lead passageway (either within the lead passageway or against circumferential walls of the lead passageway). In at least some embodiments, the retaining elements 263, 265 are positioned in the body along a plane that is transverse to the longitudinal length of the body. In at least some embodiments, the retaining elements 263, 265 are positioned in the body along a plane that is transverse to a longitudinal length of the lead passageway.

In at least some embodiments, the lead engagement portions 475, 477 define concave cutouts 481, 483, respectively, each configured to extend around a portion of the circumferential walls of the lead passageway opposite to the other concave cutout 481, 483. In at least some embodiments, the retaining element 263 includes guide features 485a,b disposed along opposing sides of the concave cutout 481. Similarly, in at least some embodiments the retaining element 265 includes guide features 487a,b disposed along opposing sides of the concave cutout 483. The guide features 485a,b and 487a,b may be useful for preventing portions of the body (which is deformable) from being pulled inward between the retaining elements 263, 265 when the retaining elements are separated from one another during transitions of the lead retainer from the retained position to the open position.

In at least some embodiments, when the lead retainer is in the biased position, the guide features 485a and 487a abut one another. In at least some embodiments, when the lead retainer is in the biased position, the guide features 485b and 487b abut one another. In at least some embodiments, when the lead retainer is in the biased position, the guide features 485a and 487a abut one another and the guide features 485b and 487b abut one another.

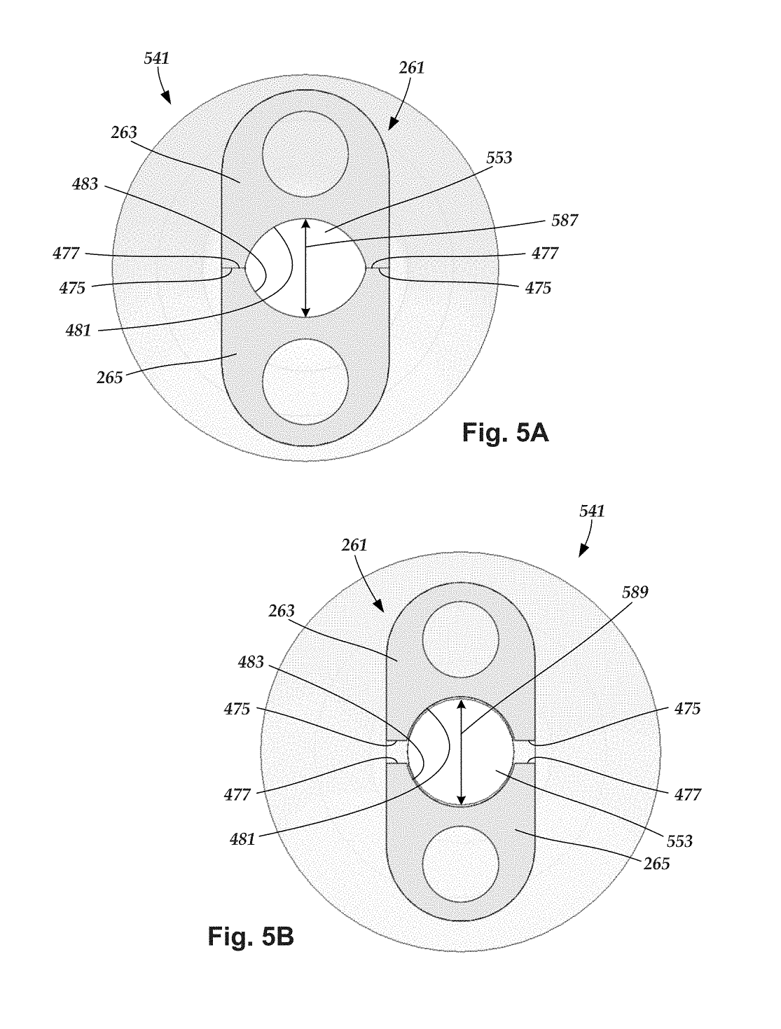

FIGS. 5A-5B show one embodiment of retaining elements 263, 265 of the lead retainer 261 in a biased position and in an open position. FIG. 5A shows, in schematic, transverse cross-sectional view, one embodiment of the lead retainer 261 disposed in a lead anchor 541 in the biased position. As shown in FIG. 5A, the lead engagement portions 475, 477 of the retaining elements 263, 265 are abutting one another and the concave cutouts 481, 483 collectively form a space along a plane transverse to the longitudinal length of the lead anchor body (or the lead passageway 553, or both) with a smallest diameter 587 that is smaller than a corresponding diameter of an inserted lead. Accordingly, the lead engagement portions of the retaining elements along the concave cutouts extend into (or cause the circumferential walls to extend into), or impinge upon, the lead passageway (see e.g., FIG. 3B). The impingement upon the lead passageway is sufficient to cause a received lead to resist axial movement along the lead passageway relative to the lead anchor.

FIG. 5B shows, in schematic, transverse cross-sectional view, one embodiment of the lead retainer 261 disposed in the lead anchor 541 and transitioned from the biased position to the open position. The lead engagement portions 475, 477 of the retaining elements 263, 265 are separated from one another by an amount sufficient to enable a received lead to move axially along the lead passageway relative to the lead anchor. In FIG. 5B, the concave cutouts 481, 483 of the retaining elements 263, 265 are separated from one another by an amount that is no less than a diameter 589 of an inserted lead.

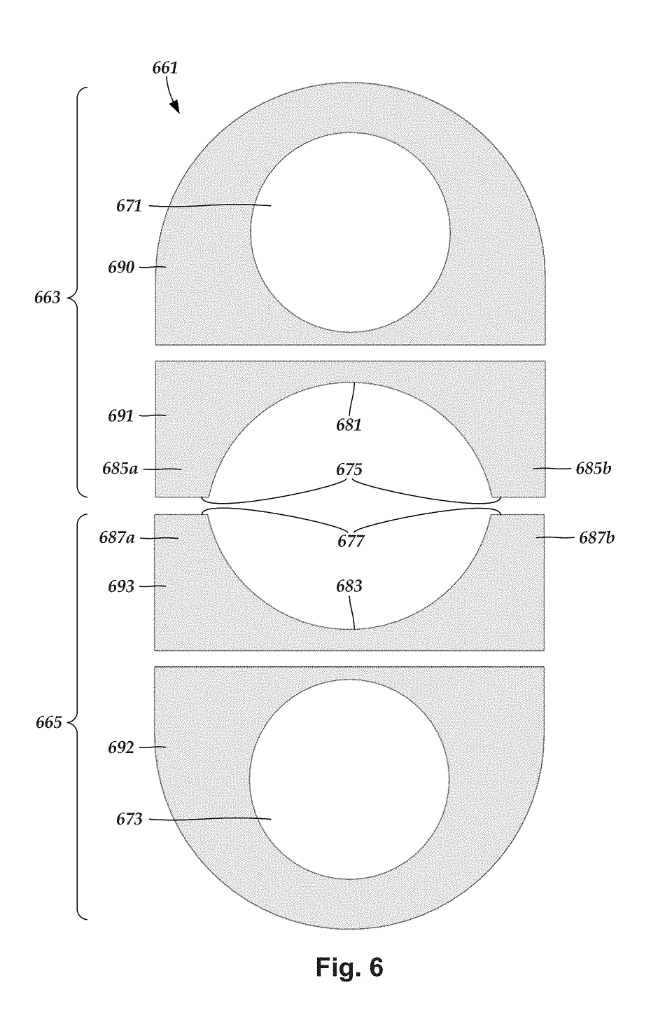

Turning to FIG. 6, separation of the retaining elements via the tool engagement apertures can cause a corresponding separation of the lead engagement portions of the lead retainer regardless of whether or not the tool engagement apertures and the lead engagement portions are positioned along the same substrate (e.g., a retaining element). In at least some embodiments, at least a portion of the lead retainer is encased in the body of the lead anchor. In which case, for example, separation of the retaining elements causes a corresponding stretching of the body which, in turn, causes the lead engagement portions to move with the retaining elements even if the lead engagement portions are not disposed on the retaining elements.

Thus, movement of the retaining elements can cause a corresponding movement of the lead engagement portions even when the lead engagement portions are not disposed along the retaining element, but rather are disposed along different substrates. Accordingly, as an alternative to the embodiment of the lead retainer shown in FIG. 4 where the retaining elements are formed as single-piece structures, in at least some embodiments the lead retainer includes retaining elements formed as multi-piece structures. In at least some embodiments, the tool engagement apertures are disposed on different pieces of the retaining elements than the lead engagement portions.

FIG. 6 shows, in schematic end view, another embodiment of a lead retainer 661 suitable for placement within either the lead anchor 241 or the lead anchor 341 in lieu of (or in addition to) the lead retainer 261. The lead retainer 661 includes a first retaining element 663 and a second retaining element 665. The retaining elements 663, 665 include the same features (e.g., tool engagement apertures, lead engagement portions, optional concave cutouts, optional guide features, and the like) and general layout as the lead retainer 261. The retaining elements 263, 265, however, include multiple pieces so that the tool engagement apertures are disposed on different structures from the lead engagement portions.

The first retaining element 663 includes a first tool engagement piece 690 and a first lead engagement piece 691. A first tool engagement aperture 671 is defined along the first tool engagement piece and a first lead engagement portion 675 is disposed along the first lead engagement piece. Similarly, the second retaining element 663 includes a second tool engagement piece 692 and a second lead engagement piece 693. A second tool engagement aperture 673 is defined along the second tool engagement piece and a second lead engagement portion 677 is disposed along the second lead engagement piece.

In at least some embodiments, the lead engagement portions 675, 677 define concave cutouts 681, 683, respectively, each configured to extend around a portion of the lead passageway opposite to the other concave cutout 681, 683. In at least some embodiments, the first lead engagement portion 691 includes guide features 685a,b disposed along opposing sides of the concave cutout 481. Similarly, in at least some embodiments the second lead engagement portion 693 includes guide features 687a,b disposed along opposing sides of the concave cutout 683.

It may be advantageous to form the retaining elements 663, 665 as multiple pieces. Doing so enables the lead engagement pieces and the tool engagement pieces to be formed from different materials, or made in different thicknesses, strengths, or sizes. In other embodiments, the retaining elements are further broken down into additional pieces.

Turning to FIGS. 7A-7B, any suitable tool can be used for transitioning the lead retainer from the biased position to the open position. In at least some embodiments, the tool includes separable probes of sufficient size for accessing the tool engagement apertures via the tool lumens, and sufficient strength for implementing an applied force for counteracting the bias of the lead retainer for maintaining the biased position. In at least some embodiments, the tool is handheld.

FIG. 7A shows, in schematic perspective view, one embodiment of a tool 792 suitable for transitioning a lead retainer, such as the lead retainer 261 or 661, from the biased position to the open position. The tool 792 is formed as a handheld, forceps-style hinged instrument that includes a first member 794a attached to a second member 794b by a hinged connection 795. The first member 794a includes a handle 796a disposed along a first portion of the hinged connection 795 and a probe 798a disposed along a second portion of the first member 794a opposite the hinged connection from the first portion. Similarly, the second member 794b includes a handle 796b along a first portion of the hinged connection 795 and a probe 798b disposed along a second portion of the second member 794b opposite the hinged connection from the first portion. FIG. 7B shows one embodiment of the probes 798a,b in close-up perspective view.

The hinged connector can be configured to move the probes in either the same direction as movement of the handles, or in a direction opposite to the movement of the handles. In FIG. 7A, the hinged connector 795 is shown configured to move the probes 798a,b in a direction opposite to the movement of the handles 796a,b. As shown by directional arrows, inward movement 797a,b of the handles 796a,b, respectively, relative to one another causes a corresponding outward movement 799a,b, respectively, of the probes 798a,b relative to one another.

It may be an advantage to have the probes move in a direction opposite to the movement of the handles so that separation of the lead retainer is a squeezing motion. Additionally, the embodiment of the tool 792 shown in FIGS. 7A-7B includes bent members 794a,b. Utilizing bent members can also be an advantage to increase ergonomics during squeezing of the handles.

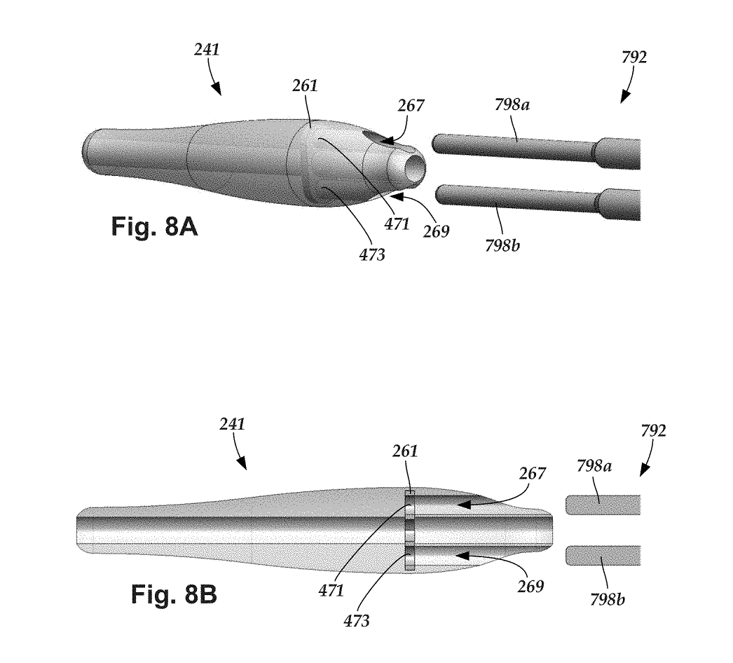

FIG. 8A shows, in schematic perspective view, one embodiment of the lead anchor 241 and the probes 798a,b of the tool 792. FIG. 8B shows the lead anchor 241 and the probes 798a,b of the tool 792 in longitudinal, cross-sectional view. As shown in FIGS. 8A-8B, the probes 798a,b are aligned with the tool lumens 267, 269, respectively. As described above, the tool lumens 267, 269 extend at least to the tool engagement apertures 471, 473, respectively, of the lead retainer 261 disposed in the lead anchor 241.

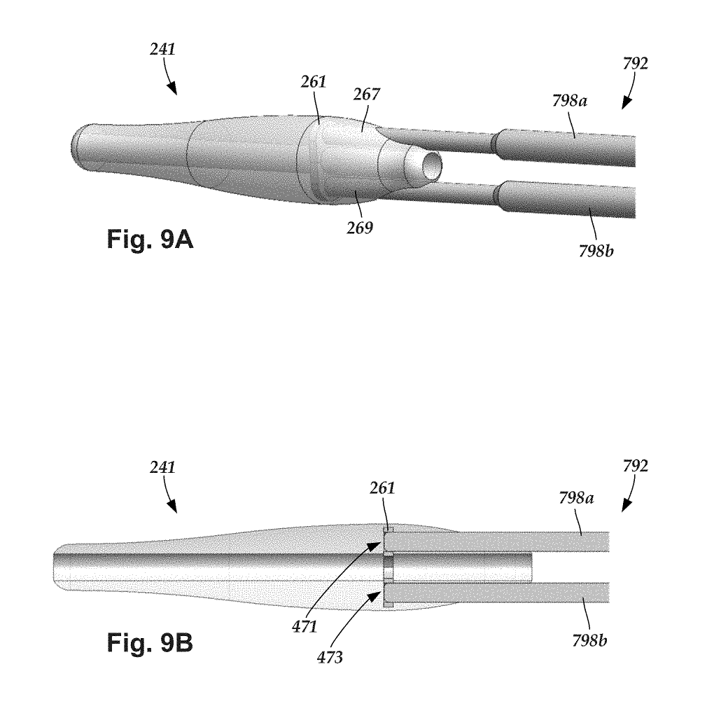

FIG. 9A shows, in schematic perspective view, one embodiment of the probes 798a,b of the tool 792 inserted into the lead anchor 241. FIG. 9B shows the probes 798a,b of the tool 792 inserted into the lead anchor 241 in longitudinal, cross-sectional view. As shown in FIGS. 9A-9B, the probes 798a,b are inserted into the tool lumens 267, 269, respectively, and extend to the tool engagement apertures 471, 473, respectively, of the lead retainer 261 disposed in the lead anchor 241.

Returning briefly to FIG. 7A, as shown by directional arrows 797a,b, bringing the handles 796a,b together causes the probes 798a,b to separate from one another (as shown by directional arrows 799a,b). Separation of the probes from one another while the probes are extended through the tool engagement apertures, as shown in FIGS. 9A-9B, separates the retention elements of the lead retainer, thereby transitioning the lead retainer from the biased position to the open position.

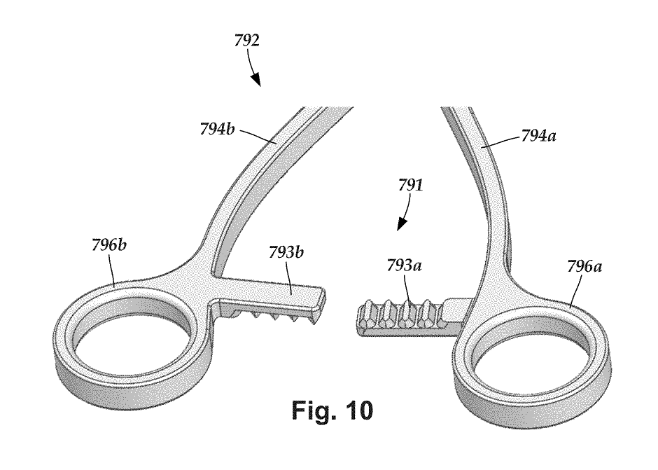

Turning to FIG. 10, in at least some embodiments the retaining element is biased in the biased position by the deformable (e.g., elastic) body. Accordingly, application of some amount of force is needed to counteract the bias (i.e., stretch the body) to transition the retaining element to an open position. It may be advantageous to be able to maintain the retaining element in an open position for an extended period of time (e.g., enough time to thread a lead through the lead anchor) without continuously applying the counteracting force manually.

FIG. 10 shows, in schematic perspective view, one embodiment of a portion of the tool 792 that includes an optional ratcheting mechanism 791 suitable for enabling the tool, when inserted into the tool engagement apertures of the lead retainer, to be maintained in a position suitable for keeping the lead retainer in an open position until manually released. The ratcheting mechanism 791 includes a first ratchet 793a extending from the first member 794a configured to releasably couple with a second ratchet 793b extending from the second member 794b. In FIG. 10, the ratcheting mechanism 791 is disposed in proximity to the handles 796a,b and is releasable by lateral movement of the first and second members relative to one another (i.e., in a movement orthogonal to the directional arrows 797a,b of FIG. 7A).

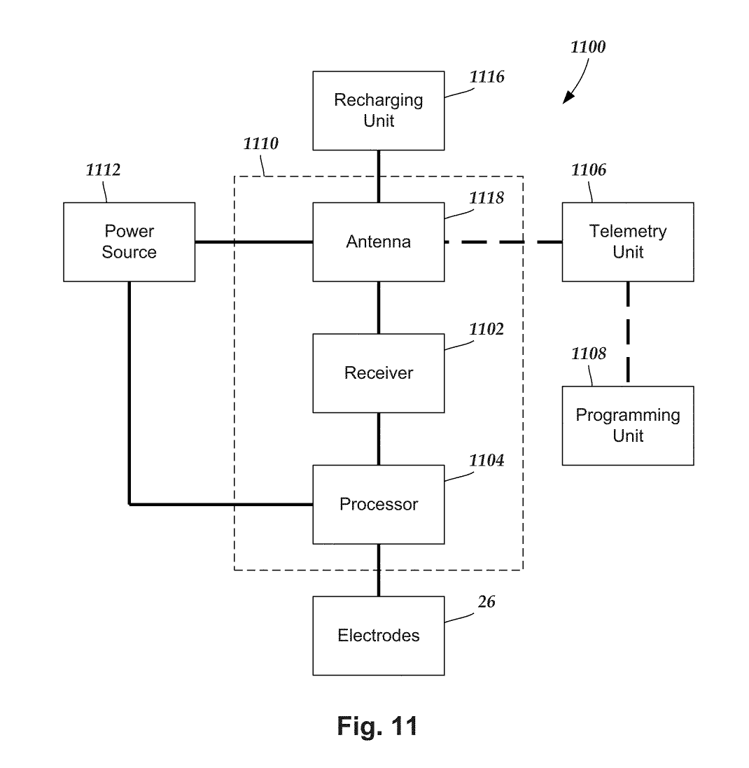

FIG. 11 is a schematic overview of one embodiment of components of an electrical stimulation system 1100 including an electronic subassembly 1110 disposed within a control module. It will be understood that the electrical stimulation system can include more, fewer, or different components and can have a variety of different configurations including those configurations disclosed in the stimulator references cited herein.

Some of the components (for example, a power source 1112, an antenna 1118, a receiver 1102, and a processor 1104) of the electrical stimulation system can be positioned on one or more circuit boards or similar carriers within a sealed housing of an implantable pulse generator, if desired. Any power source 1112 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1118 or a secondary antenna. The external power source can be in a device that is mounted on the skin of the user or in a unit that is provided near the user on a permanent or periodic basis.

If the power source 1112 is a rechargeable battery, the battery may be recharged using the optional antenna 1118, if desired. Power can be provided to the battery for recharging by inductively coupling the battery through the antenna to a recharging unit 1116 external to the user. Examples of such arrangements can be found in the references identified above.

In one embodiment, electrical current is emitted by the electrodes 26 on the paddle or lead body to stimulate nerve fibers, muscle fibers, or other body tissues near the electrical stimulation system. The processor 1104 is generally included to control the timing and electrical characteristics of the electrical stimulation system. For example, the processor 1104 can, if desired, control one or more of the timing, frequency, strength, duration, and waveform of the pulses. In addition, the processor 1104 can select which electrodes can be used to provide stimulation, if desired. In some embodiments, the processor 1104 selects which electrode(s) are cathodes and which electrode(s) are anodes. In some embodiments, the processor 1104 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1108 that, for example, allows modification of pulse characteristics. In the illustrated embodiment, the processor 1104 is coupled to a receiver 1102 which, in turn, is coupled to the optional antenna 1118. This allows the processor 1104 to receive instructions from an external source to, for example, direct the pulse characteristics and the selection of electrodes, if desired.

In one embodiment, the antenna 1118 is capable of receiving signals (e.g., RF signals) from an external telemetry unit 1106 which is programmed by the programming unit 1108. The programming unit 1108 can be external to, or part of, the telemetry unit 1106. The telemetry unit 1106 can be a device that is worn on the skin of the user or can be carried by the user and can have a form similar to a pager, cellular phone, or remote control, if desired. As another alternative, the telemetry unit 1106 may not be worn or carried by the user but may only be available at a home station or at a clinician's office. The programming unit 1108 can be any unit that can provide information to the telemetry unit 1106 for transmission to the electrical stimulation system 1100. The programming unit 1108 can be part of the telemetry unit 1106 or can provide signals or information to the telemetry unit 1106 via a wireless or wired connection. One example of a suitable programming unit is a computer operated by the user or clinician to send signals to the telemetry unit 1106.

The signals sent to the processor 1104 via the antenna 1118 and the receiver 1102 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1100 to cease operation, to start operation, to start charging the battery, or to stop charging the battery. In other embodiments, the stimulation system does not include the antenna 1118 or receiver 1102 and the processor 1104 operates as programmed.

Optionally, the electrical stimulation system 1100 may include a transmitter (not shown) coupled to the processor 1104 and the antenna 1118 for transmitting signals back to the telemetry unit 1106 or another unit capable of receiving the signals. For example, the electrical stimulation system 1100 may transmit signals indicating whether the electrical stimulation system 1100 is operating properly or not or indicating when the battery needs to be charged or the level of charge remaining in the battery. The processor 1104 may also be capable of transmitting information about the pulse characteristics so that a user or clinician can determine or verify the characteristics.

The above specification and examples provide a description of the manufacture and use of the invention. Since many embodiments of the invention can be made without departing from the spirit and scope of the invention, the invention also resides in the claims hereinafter appended.

What is claimed:

1. A lead anchor comprising:
an elongated body formed from a deformable material, the body having an outer surface and a longitudinal length with a first end and an opposing second end, the body defining a lead passageway configured and arranged to receive a lead, the lead passageway extending along the entire longitudinal length of the body from the first end to the second end;
a lead retainer formed from a rigid material and disposed in the body around a portion of the lead passageway, the lead retainer comprising
a first retaining element,
a second retaining element, wherein the portion of the lead passageway is between the first and second retaining elements;
a first tool lumen extending along the longitudinal length of the body between the outer surface of the body and the first retaining element; and
a second tool lumen extending along the longitudinal length of the body between the outer surface of the body and the second retaining element;
wherein the first and second retaining elements are biased to exert a compressive force against a portion of a lead disposed in the lead passageway to resist axial movement of the lead relative to the lead anchor, wherein the lead anchor is configured and arranged so that a tool inserted into the first and second tool lumens can separate the first retaining element from the second retaining element to facilitate insertion or removal of the portion of the lead from the lead passageway.

2. The lead anchor of claim 1, wherein the first and second retaining elements are formed as single-piece structures.

3. The lead anchor of claim 1, wherein the first retaining element defines a first tool engagement aperture open to the first tool lumen, and wherein the second retaining element defines a second tool engagement aperture open to the second tool lumen.

4. The lead anchor of claim 3, wherein the first tool engagement aperture is configured and arranged to receive a first probe of a tool extended along the first tool lumen, and wherein the second tool engagement aperture is configured and arranged to receive a second probe of the tool extended along the second tool lumen.

5. The lead anchor of claim 3, wherein the first retaining element comprises a first lead engagement portion, and wherein the second retaining element comprises a second lead engagement portion, the first and second lead engagement portions disposed along the lead passageway.

6. The lead anchor of claim 5, wherein the first retaining element is formed as a first multi-piece structure, and wherein the second retaining element is formed as a second multi-piece structure.

7. The lead anchor of claim 6, wherein the first multi-piece structure comprises a first tool engagement piece and a first lead engagement piece with the first tool engagement aperture defined in the first tool engagement piece and the first lead engagement portion disposed along the first lead engagement piece, and wherein the second multi-piece structure comprises a second tool engagement piece and a second lead engagement piece with the second tool engagement aperture defined in the second tool engagement piece and the second lead engagement portion disposed along the second lead engagement piece.

8. The lead anchor of claim 5, wherein the first lead engagement portion comprises a first concave cutout configured and arranged to extend around a portion of the circumference wall, and wherein the second lead engagement portion comprises a second concave cutout configured and arranged to extend around a portion of the circumferential wall circumferentially opposite to the portion of the circumferential wall around which the first concave cutout extends.

9. The lead anchor of claim 8, wherein the first lead engagement portion comprises first guide features disposed on opposing sides of the first concave cutout, and wherein the second lead engagement portion comprises second guide features disposed on opposing sides of the second concave cutout, the first and second guide features collectively configured and arranged to prevent portions of the body from being pulled inwardly between the first and second retaining element when the first retaining element is separated from the second retaining element.

10. The lead anchor of claim 3, wherein the first tool lumen terminates at, or in proximity to, the first tool engagement aperture of the lead retainer.

11. The lead anchor of claim 1, wherein the first and second tool lumens open to the outer surface of the body along the first end.

12. The lead anchor of claim 1, wherein at least one of the first tool lumen or the second tool lumen extend along the entire longitudinal length of the body and are open to the outer surface of the body along both the first and second ends.

13. An anchoring arrangement, comprising:
the lead anchor of claim 3; and
a tool comprising
a first probe configured and arranged to extend through the first tool lumen of the lead anchor body and the first tool engagement aperture of the lead retainer, and
a second probe configured and arranged to extend through the second tool lumen of the lead anchor body and the second tool engagement aperture of the lead retainer.

14. The anchoring arrangement of claim 13, further comprising an electrical stimulation lead comprising a plurality of electrodes, wherein the lead anchor is configured and arranged to receive a portion of the electrical stimulation lead and removably retain the received portion of the electrical stimulation lead.

15. An implantable stimulation arrangement, comprising:
the lead anchor of claim 1; and
an electrical stimulation lead comprising a plurality of electrodes, wherein the lead anchor is configured and arranged to receive a portion of the electrical stimulation lead and removably retain the received portion of the electrical stimulation lead.

16. An implantable stimulation system, comprising:
the lead anchor of claim 1;
an electrical stimulation lead comprising a plurality of electrodes, the electrical stimulation lead coupleable to the lead anchor; and
a control module coupleable to the electrical stimulation lead.

17. A method of anchoring a lead, comprising:
providing the lead anchor of claim 1;
advancing an electrical stimulation lead to a target stimulation location within a patient;
applying a force to separate the first and second retaining elements of the lead retainer of the lead anchor, the applied force counteracting an opposing compressive force exerted by the body of the lead anchor;
inserting the electrical stimulation lead into the lead passageway and advancing the electrical stimulation lead relative to the lead anchor while applying the force to separate the first and second retaining elements; and
releasing the applied force to hold the electrical stimulation lead in position relative to the lead anchor by the compressive force exerted by the lead retainer.

18. The method of claim 17, wherein applying a force to separate the first and second retaining elements comprises:
inserting a first probe of a tool through a first tool engagement aperture of the first retaining element of the lead retainer;
inserting a second probe of the tool through a second tool engagement aperture of the second retaining element of the lead retainer; and
using the probes to physically separate the first and second tool engagement apertures from one another.

19. The method of claim 18, wherein inserting a first probe of a tool through the first tool engagement aperture comprises advancing the first probe along the longitudinal length of the lead anchor within the first tool lumen; and wherein inserting a second probe of a tool through the second tool engagement aperture comprises advancing the second probe along the longitudinal length of the lead anchor within the second tool lumen.

20. The method of claim 17, further comprising anchoring the lead anchor to patient tissue.
